(19) United States
(12) Patent Application Publication
(10) Pub. No.: US 2017/0128271 A1
(43) Pub. Date: May 11, 2017

(54) THERMALLY CONDUCTIVE MATERIAL-INFUSED HYDROGEL BANDAGES

(71) Applicant: ADVANCED FIRST AID RESEARCH PTE. LTD., SG (SG)

(72) Inventors: Carl J. FREER, SG (SG); Joseph M. MARTEN, SG (SG); Stephen J. CARROLL, Liverpool (GB); Zhilong SHI, SG (SG)

(73) Assignee: ADVANCED FIRST AID RESEARCH PTE. LTD., SG (SG)

(21) Appl. No.: 15/317,438

(22) PCT Filed: Jun. 13, 2014
(86) PCT No.: PCT/SG2014/000282

§ 371 Date: Dec. 9, 2016

(51) Int. Cl. A61F 13/02; A61L 15/18; A61F 13/00; A61L 15/20; A61L 15/26; A61L 15/60; A61L 15/24

(52) U.S. Cl. CPC A61F 13/0213; A61L 15/60; A61L 15/18; A61L 15/24; A61L 15/20; A61L 15/26; A61F 13/0226; A61F 13/00059; A61F 2013/00157; A61F 2013/00187

(57) ABSTRACT

The invention is a class of medical bandages that are effective for use in the treatment of various types of tissue burns, such as burns due to heat, chemicals, or sun exposure. The inventive bandages are comprised of a thin and flexible heat sink such as a hydrogel infused with a thermally conductive material such as aluminum oxide particles. The inventive bandages rapidly cool a burn wound by ensuring direct contact of the infused hydrogel with the burn to draw heat away from the burn via conduction.

### BACKGROUND OF THE INVENTION

Burn injuries are caused by heat, chemicals, electricity, radiation, and friction and can vary in severity. First degree (superficial) burns are the least severe, causing redness, and healing relatively quickly. On the other end of the spectrum, fourth degree burns are the most severe, burning down to the level of the muscle and bone. Second (partial thickness) and third degree (full thickness) burns fall between these extremes.

Medical professionals often try to strike a balance when deciding how to treat burns. On one hand, if a burn is superficial and relatively dry, then it may be desirable to keep the wound moist with water or some sort of ointment or cream. However, a problem with applying many ointments and/or creams is that such applications often do not help, or worse even prevent drawing heat away from a burn. On the other hand, if a burn is more serious, such as a second-degree burn that is oozing fluid, then there is an enhanced fear of infection. In such cases, some medical professionals feel that such wounds should be kept relatively dry, while still others may advocate for the application of various ointment dressings with antibiotic properties to fight infection. Hence, it would be desirable to come up with a treatment strategy that is able to provide the best of all worlds.

Bandages and wraps may incorporate a thin layer of thermally conductive metal (such as aluminum) at the base of a substrate adapted to be in direct contact with a burn wound, while the top side of the aluminum substrate has a heat-dissipation-enhancing topography to help cool burns faster by enhancing thermal convection properties. Such products are described in U.S. Pat. No. 8,530,720 to Freer, et al. Heat from a burn will be drawn from the burn to the metal substrate through conduction. Aluminum does not effectively store conducted heat but is an excellent conductor of heat. Aluminum conducts heat away from the source and readily gives the heat up to its surrounding atmospheric environment through convection.

Certain heat-dissipation-enhancing-topographies of the thermally conductive layer may have technically complicated designs or may be difficult to manufacture—cheaply or efficiently. In situations where one wishes to reduce the complexity of the thermally conductive metal's topography, it may be desirable to incorporate into a thermally conductive bandage a more efficient method of heat transfer away from a burn via conduction, rather than convection. When one side of a thermally conductive bandage is applied to a burn, an additional layer of material may be present on the opposite side of the conductive bandage substrate to act as a heat sink. This additional layer will act as a heat sink into which heat can be removed from the burn area and stored, or further dissipated into the atmospheric environment through convection. Hydrogel may act as a convenient heat sink in such applications.

An alternative to having two discrete layers (a thermally conductive layer and a heat sink layer) would be to have a single layer having properties of a thermally conductive layer as well as properties of a heat sink layer. Such a layer may be formed as a hydrogel heat sink with a thermally conductive material, such as aluminum oxide (alumina) microparticles, incorporated into the hydrogel.

It would be advantageous to develop a bandage having aluminum oxide microparticles or other thermally conductive materials in combination with a hydrogel substrate having enhanced conductivity so that heat may be rapidly drawn away from the burn by the thermally conductive materials and into the hydrogel heat sink. Such a bandage would effectively cool a subject's burn and further alleviate pain associated with subject's burn.

### SUMMARY OF THE INVENTION

The invention is a class of medical products designed to alleviate discomfort and relieve pain caused by burns. The inventive bandage includes a layer of a heat sink (particularly a hydrogel) infused with a thermally conductive material (particularly aluminum oxide particles). A bandage incorporating a hydrogel as a primary cooling agent infused with aluminum oxide particles would improve thermal transfer from a subject's burn to a thermal heat sink.

Hydrogels are networks of hydrophilic polymer chains in which water is the dispersion medium. Some hydrogels have over 99% water. Hydrogels generally exhibit flexibility similar to that of human tissue due to their substantial water content. Water has a high specific heat capacity, and a hydrogel having a large water content will similarly have a high specific heat capacity. High specific heat capacity, coupled with physical flexibility and biocompatible nature, make hydrogels a preferred choice for a heat sink in the inventive bandage.

Maximizing the thermal conductivity and specific heat capacity reservoir increases the rate of cooling. The thermally conductive material infused throughout the hydrogel ensures flexibility and effective heat-transfer characteristics to rapidly cool a burn wound. The thermally conductive material facilitates heat transfer from the wound via conduction through the hydrogel which acts as a thermal reservoir, as a humectant, and helps to cool a burn.

The inventive bandage is secured over a subject's burn with a top layer of adhesive material adapted for use on the subject. A removable backing layer adhered to the very bottom of the bandage protects the adhesive material and the burn-contacting portions of the bandage until the backing layer is removed from the bandage for use. It is preferred that the bandage components are thin and flexible to enhance patient comfort. It is preferred that the bandages assist with wound healing and provide an environment to help control fluid loss, protect against abrasion, friction, desiccation, and contamination.

Methods of using the inventive bandage include facilitating and expediting heat-dissipation from a burn to assist in the healing of a burn. It is a goal of the invention to return the area of the wound to normal skin temperature within about fifteen (15) to about 300, more preferably within about 15 to about 120 seconds. It is a goal of the invention to alleviate, reduce, and eliminate symptoms of burn within about fifteen (15) to about 300 seconds of bandage application.

An alternative embodiment of the inventive bandage includes an additional conductive substrate (preferably aluminum sheet) directly onto the subject's burn to draw heat away from the burn through conduction. Bonded to the opposite side of the conductive substrate is the aluminum-infused hydrogel which draws heat away from the conductive substrate (aluminum sheet).

These and other features, aspects and advantages of the present invention will become better understood with reference to the following drawings, description, and claims.

### BRIEF DESCRIPTION OF THE DRAWINGS

FIG. 1 shows an expanded assembly of a bandage including a bottom backing layer, thermally conductive material-infused hydrogel absorber layer, and top adhesive layer.

FIG. 2 shows a cut-away schematic of an assembled bandage of FIG. 1 indicating the position and sizes of the layers.

FIG. 3 depicts a cross-sectional view of the layers within the assembled bandage shown in FIG. 2.

FIG. 4 shows an expanded assembly of a bandage including a bottom backing layer, metal thermal radiator layer, hydrogel absorber layer, and top adhesive layer.

FIG. 5 shows a cut-away schematic of an assembled bandage of FIG. 4 indicating the position and sizes of the layers.

FIG. 6 depicts a cross-sectional view of the layers within the assembled bandage shown in FIG. 5.

### DETAILED DESCRIPTION OF THE INVENTION

There are three ways in which thermal energy transfer can be described: Conduction; Convection; and Radiation. Conduction requires physical contact (similar to the flow of electricity in wire). Convection emanates from the movement of molecules (e.g., the way in which heated and cooled water or other fluid moves up and down). Radiation does not necessarily involve direct contact (e.g., the way the sun emits light rays).

At any given temperature, a given mass of aluminum holds much less energy than an equivalent mass of human flesh. For instance, in convection or conduction, if one touches aluminum foil from an oven during the cooking process, a subject's hand and the foil share the thermal energy. The hand (of much greater mass) requires much more energy to raise its temperature (if at all, depending upon the physical connection between the foil and the food). When the subject touches aluminum foil, the foil transfers heat to the flesh; however, due to the aluminum's low specific-heat capacity, the foil quickly loses energy, barely raising the temperature of the skin in contact. Because aluminum foil does not effectively store conducted heat it therefore facilitates the “cooling” of a burn.

Aluminum is non-toxic and used widely in the medical industry. While aluminum does not effectively store conducted heat, aluminum is nonetheless an excellent conductor of heat. Aluminum conducts heat away from the source and readily gives the heat up to its surroundings. This has a cooling effect to the source of the heat. Aluminum can be an effective conductor of a subject's body heat, alleviating pain which emanates from added warmth on a subject's burn. Aluminum metal is generally unreactive and non-toxic, and aluminum will resist adhering to a burn wound—these properties permit aluminum to conduct heat away from the burn without negatively interfering with natural wound healing processes.

Convection generally has significantly lower thermal transfer effects than conduction. Conduction can transfer hundreds or even thousands of times more thermal energy than convection. For planar wall conduction—when the non-controllable variables are removed—the thermal transfer is directly proportional to the thermal conductivity multiplied by the contact area, divided by the wall thickness. For convection—when the non-controllable variables are removed—the thermal transfer is directly proportional to the contact area. Minimizing material thickness and optimizing thermal conductivity are expected to transfer thermal energy at a rate thousands of times faster through conduction than via convection.

The bandages of the invention utilize a layer of a heat sink infused with a thermally conductive material to draw heat away from the burn via conduction. The inventive bandages are designed to swiftly and efficiently alleviate discomfort and pain caused by burns including those resulting from sun exposure, fire, chemicals, electricity, or friction.

The inventive bandages contain a heat sink infused with a thermally conductive material. A preferred heat-sink is a hydrogel substrate that is flexible, biocompatible, and acts as a thermal reservoir. The hydrogel ideally is tacky and exhibit a moderate adhesiveness to the wound and surrounding skin, but not to the new forming dermis, to help hold the bandage in place. Hydrogel may act as a convenient heat sink in the inventive bandages in part because of the high specific heat of water (4.18 Joules/(grams×degree Kelvin)). Preferred hydrogels have a high water content and a high specific heat capacity. One preferred hydrogel contains glycerol and water. In one embodiment, the hydrogel has a specific heat capacity of greater than about 2 Joules/(grams×degree Kelvin). In one embodiment, the hydrogel has a specific heat capacity of greater than about 3 Joules/(grams×degree Kelvin). In one embodiment, the hydrogel has a specific heat capacity of greater than about 4 Joules/(grams×degree Kelvin).

The hydrogel substrate is preferably sized as a thin sheet. Maximizing the thermal conductivity and specific heat capacity of the hydrogel thermal reservoir increases the rate of cooling, but as the hydrogel thickness is increased the bandage will increase in rigidity. However, as the hydrogel thickness is reduced, thermal capacity may be reduced. The hydrogel substrate is preferably in the range from about 0.005 inches to about 0.100 inches thick. In one embodiment the hydrogel layer is about 0.005 inches to about 0.050 inches thick. The hydrogel may be about 0.005 inches, about 0.010 inches, about 0.015 inches, about 0.020 inches, about 0.025 inches, about 0.030 inches, about 0.035 inches, about 0.040 inches, about 0.045 inches, about 0.050, about 0.055 inches, about 0.060 inches, about 0.065 inches, about 0.070 inches, about 0.075 inches, about 0.080 inches, about 0.085 inches, about 0.090 inches, about 0.095 inches, or about 0.100 inches in thickness. In one embodiment, the hydrogel is about 0.030 inches thick. In one embodiment, the hydrogel layer is about 0.015 inches thick. In one embodiment, the hydrogel layer is about 0.010 inches thick.

The hydrogels of the inventive bandages may be prepared by in situ monomer polymerization in the presence of a multifunctional monomer (as crosslinker) or by crosslinking polymers by a variety of physical and chemical methods including heating, cooling, freeze-thaw cycles and ions crosslinking. A preferred method of making the hydrogels of the inventive bandages herein is by crosslinking monomer with ultraviolet (UV) or ionizing radiation in the presence of crosslinker and photoinitiator. Another preferred method of making the hydrogels of the inventive bandages herein is by crosslinking polymer with ultraviolet (UV) or ionizing radiation which avoids presence of any residual monomer in the finished product, and facilitates production of hydrogel in roll or sheet form. Another method of making hydrogel is thermally-reactively cured karaya in roll or sheet form.

In one embodiment, a polyvinyl alcohol (PVA) hydrogel can be produced by freeze/thaw cycles. In one embodiment agar, karaya, or gelatin in an aqueous solution can form a hydrogel after cooling the aqueous solution. In one embodiment an alginate solution can gel by adding multivalent ions of opposite charge (such as calcium ions). In other embodiments crosslinking poly(acrylic acid) (PAA) with aluminum glycinate or crosslinking polyvinyl alcohol (PVA) with borax can form a hydrogel. In other embodiments mixing solutions of a polyanion and a polycation to form a complex can form a hydrogel.

Ultraviolet (UV) radiation is safer, portable, and less expensive than high-energy radiation and is a practical alternative to ionizing radiation. Thermally conductive materials with or without surface modification can be easily incorporated into a gel by dissolving or suspending them in a polymer solution prior to UV irradiation. Inclusion of glycerol in the swelling medium may increase the moisture maintenance of hydrogel and permeability of biological membranes to drugs. In one embodiment a monomer such as acrylamide or acrylic acid is used with a bi-functional monomer N,N′-methylene-bisacrylamide or polyethylene glycol diacrylate (PEGDA) as crosslinker, and a photoinitiator is used for UV irradiated polymerization, for example, Irgacure 2959 (1-[4-(2-Hydroxyethoxy)-phenyl]-2-hydroxy-2-methyl-1-propane-1-one). Hydrogels made in this manner using UV radiation may be formed as a roll.

Polyurethane and silicone hydrogels may also be used. However, these may be more expensive, have lower thermal conductivity, and/or shorter term of moisture maintenance compared with other embodiments described herein.

In a preferred embodiment, ionizing radiation is used to crosslink polymers for making the hydrogel. It is preferred to manufacture the hydrogel starting with a non-toxic, biologically compatible, high molecular weight polymer and then cross-linking the polymers with ionizing radiation. Ionizing radiation, such as gamma or electron beam, is a convenient method for cross-linking polymers while simultaneously sterilizing the product. A dose of 25 kGy is normally sufficient to sterilize material and ensure the formation of a stable, cross-linked hydrogel. Crosslinking reactions lead to the formation of hydrogel in which individual polymer chains are connected by stable covalent bonds. Preferred embodiments using ionizing radiation to form a hydrogel include polyethylene oxide (PEO), carboxymethyl cellulose (CMC), polyvinyl alcohol (PVA), and polyvinylpyrrolidone (PVP) and mixtures thereof. In a preferred embodiment the polymer comprises polyvinylpyrrolidone (PVP); in a preferred embodiment the hydrogel comprises about 8% polyvinylpyrrolidone (PVP). Hydrogels made in this manner using ionizing radiation may be formed as a roll.

In one embodiment, hydrogel substrates for use in the present invention may be comprised of ingredients and components well-known in the art and may be formed according to a variety of methods, modified appropriately to incorporate the thermally conductive material into the hydrogel. In one embodiment the hydrogel substrate is made using methods and components described in Sekisui Plastics Co., Ltd. European Patent Application EP 2662429A1 paragraphs 0008, 0011, 0013, 0016, 0020, and 0026 which are incorporated herein by reference. In one embodiment the hydrogel is prepared as a sheet.

Additional components of the hydrogel may include: polyacrylate sodium, propylene glycol, dipropylene glycol, diglycerin, glycerin, magnesium aluminum silicate, tartaric acid, butylene glycol, glycerol, and polyglyceryl-6 laureate. In one embodiment, the hydrogel comprises: water, aluminum oxide particles, polyacrylate sodium, glycerol, tartaric acid, magnesium aluminum silicate, and polyglyceryl-6 laureate.

Thermally conductive materials that may be infused in the heat sink layer include, for example, metals, metal oxides, alloy, ceramics, carbon-based materials, and composites thereof. Preferred thermally conductive material includes aluminum and aluminum oxide (Al₂O₃), titanium dioxide and zinc oxide (ZnO). Other thermally conductive material includes aluminum nitride, aluminum hydroxide, clay, magnesium oxide, gold, silver, copper, yttrium oxide, iridium, calcium and calcium compounds, silicon, silicon carbide, silicon nitride, silicon dioxide, zinc, zinc oxide, titanium, titanium dioxide, tungsten, graphite, graphene, diamond, C60, carbon fiber, carbon nanotubes, and graphene oxide. Still other thermally conductive material includes iron, iron oxide, nickel, tin, palladium, silver oxide, copper oxide, and tin oxide. Thermally conductive fibers, strips, or fabrics may be used such as carbon fiber, cotton fabric, glass fibers, or non-woven polyester fabric. One thermally conductive material or more than one thermally conductive material may be used. In one embodiment aluminum oxide is used as a thermally conductive material in the heat sink layer; in one embodiment aluminum oxide and titanium dioxide are used as thermally conductive materials in the heat sink layer.

The thermally conductive material may be sized and dispersed throughout the hydrogel layer in any manner that does not compromise the physical integrity of the hydrogel. In one embodiment the thermally conductive material is dispersed evenly throughout the hydrogel; in one embodiment the thermally conductive material is dispersed as a gradient across the hydrogel with the thermally conductive material located substantially towards one surface. In one embodiment the hydrogel comprises two or more layers each having a different thermally conductive material dispersed in each layer.

Thermally conductive material may be sized, for example, as particles such as nanoparticles or microparticles, or fibers (such as carbon fiber or silicon carbide fiber) or tubes (such as carbon nanotube) or sheets (such as graphene and graphene oxide). The thermally conductive material may comprise particles having a distribution of sizes or particles having relatively uniform size. Thermally conductive material utilized in the invention may be sized on the nanometer, micrometer, or millimeter scale. The thermally conductive material utilized in the invention is preferably sized as microparticles (diameter larger than about 0.1 micrometers and preferably between about 5 to about 500 micrometers) or nanoparticles (diameter smaller than about 100 nanometers). The thermally conductive material is preferably in the range from about 1 nanometer to about 500 micrometers, preferably about 5 micrometers to about 500 micrometers. In one embodiment the thermally conductive material comprises particles having a mean size of approximately 3 to approximately 20 micrometers. In one embodiment the thermally conductive material comprises particles having a mean size of approximately 10 micrometers. In one embodiment, the aluminum oxide particles are smaller than about 100 nanometers, in another embodiment the aluminum oxide particles are about 100 nanometers. In one embodiment the aluminum oxide particles have a mean size of approximately 10 micrometers; in one embodiment, the aluminum oxide particles are approximately 10 micrometers.

Unless otherwise stated herein, percentages are reported as weight/weight. Hydrogels of the inventive bandages may be formed in a solution and may comprise the following components: monomer (about 1% to about 70%) and/or polymer (about 1% to about 30%), cross-linker (about 0.01% to about 20%), photoinitiator (about 0.001% to about 1%), thermally conductive material (about 1% to about 40%), and humectant (about 1% to about 30%). In one embodiment, the polymer may be polyvinylpyrrolidone (PVP, about 10%), the thermally conductive material may be aluminum oxide (Al₂O₃, about 10%), the humectant may be glycerol (about 10%), in a water solution. Crosslinking may be performed using ionizing radiation. In one embodiment a cross-linker may be polyethylene glycol diacrylate (PEGDA) and a photoinitiator may be 1-[4-(2-Hydroxyethoxy)-phenyl]-2-hydroxy-2-methyl-1-propane-1-one (e.g., Irgacure 2959) or 2-hydroxy-1-[4-(2-hydroxyethoxy)-phenyl]-2-methyl-1-propanone.

In one embodiment, the monomer may be acrylate or acrylamide (about 30%), the cross-linker may be methylene-bisacrylamide (about 0.1%), the photoinitiator may be 1-[4-(2-Hydroxyethoxy)-phenyl]-2-hydroxy-2-methyl-1-propane-1-one (e.g., Irgacure 2959, about 0.08%) [2-hydroxy-1-[4-(2-hydroxyethoxy)-phenyl]-2-methyl-1-propanone], the thermally conductive material may be aluminum oxide (Al₂O₃, about 10%), the humectant may be glycerol (about 10%), in a water solution. Crosslinking may be performed using UV or ionizing (gamma) radiation. In one embodiment, the monomer is 2-acrylamido-2-methylpropane sulfonic acid (AMPS, about 40%), the cross-linker is polyethylene glycol diacrylate (PEGDA, about 0.2%), the photoinitiator may be 1-[4-(2-Hydroxyethoxy)-phenyl]-2-hydroxy-2-methyl-1-propane-1-one (e.g., Irgacure 2959, about 0.05%) [2-hydroxy-1-[4-(2-hydroxyethoxy)-phenyl]-2-methyl-1-propanone], the thermally conductive material is aluminum oxide (Al₂O₃, about 10%) the humectant is glycerol (about 10%), in a water solution. Crosslinking may be performed using UV or ionizing (gamma) radiation.

Roll forming of a hydrogel is generally known in the art and comprises three components—a conveyor belt (unwind/rewind system), a curing system, and a filler or coating system with a blade to control hydrogel thickness. In one embodiment, a scrip is drawn trough a trough of uncured hydrogel liquid and out through an aperture and under a blade or gate to adjust the thickness of the hydrogel coating onto a web (e.g. a 5 mil polyethylene terephthalate sheet); this passes through a UV ionizing radiation or thermal curing system, then a cooling section, and on to a take-up station where a polyethylene film may be attached prior to rolling up the hydrogel sheet.

The hydrogel layer of the inventive bandage is coupled to a top adhesive layer which extends beyond the boundaries of the hydrogel layer. The top adhesive layer is a thin film and may be made of a polymeric material. The top adhesive layer has adhesive material disposed on the bottom surface to facilitate coupling to a subject's skin, and a top surface that is adhesive-free. Polymer medical tape may be used as the top adhesive layer. A selection of materials commonly used in medical bandages may be used as the top adhesive layer. A perforated polymer such as 1527-ENP ethylene vinyl acetate (EVA) is preferred in one embodiment. In one embodiment commercially available medical tape is used as the top adhesive layer. In one embodiment, a medical tape is made of polyethylene and poly(acrylic acid) (PAA).

A removable bandage-backing layer, or release liner, is disposed across the entire bottom surface of the bandage and is coupled to the bandage via the adhesive present in the top adhesive layer. The removable backing layer is detachably coupled to the adhesive top layer so as to be readily peeled away from the bandage. In one embodiment the backing layer extends slightly beyond the boundaries of the top adhesive layer; in one embodiment the backing layer has substantially the same surface area as the top adhesive layer and the backing layer is positioned to be flush with the top adhesive layer. The backing layer is made of a material that can adhere to the top adhesive layer during manufacturing, packaging, and storage, yet can be readily removed from the bandage when desired so as to free the bandage for application to a subject's burn.

In one embodiment the backing layer comprises two or more sheets. In one embodiment, the backing layer consists of two partially overlapping sheets. In this embodiment the each sheet may be partially in contact with the bandage, and partially in contact with the other sheet.

The release liner may be made of any appropriate material or composite, including for example kraft paper, glassine paper, polyethylene, polypropylene, or polyester. The liner may be coated, and preferably with release agents such as silicones or fluorochemicals. In a preferred embodiment the release liner is paper coated with polyethylene and silicone in one side.

In one embodiment, the top adhesive layer and hydrogel substrate of the inventive bandage are concentric to one another. In another embodiment, the hydrogel substrate is positioned so as to be off-center from the top adhesive layer within the inventive bandage. In one embodiment the entire top surface of the hydrogel substrate is in contact with a portion of the bottom surface of the top adhesive layer. In one embodiment the backing layer is in contact with the bottom surface of the inventive bandage such that the backing layer contacts a portion of the bottom surface of the top adhesive layer and the entire bottom surface of the hydrogel substrate.

The inventive bandage may take a variety of forms. In a preferred embodiment the inventive bandage is substantially rectangular; in another embodiment the inventive bandage is substantially square. In one embodiment the inventive bandage is substantially elliptical; in another embodiment the inventive bandage is substantially ovular; in yet another embodiment the inventive bandage is substantially circular. In one embodiment the inventive bandage is substantially triangular; in one embodiment the inventive bandage is substantially trapezoidal. In one embodiment the inventive bandage is substantially heart-shaped. In yet another embodiment the inventive bandage is substantially octagonal. The inventive bandage may be bow-tie shaped or butterfly shaped. The inventive bandages may have corners that are squared or rounded.

The inventive bandage may be shaped to conform to different body contours and body parts such as a glove- or mitt-shape for comfortable use on a burned hand, or an H-shaped bandage to wrap comfortably around a burned finger. The inventive bandage form-factor may be adapted to facilitate application to a part of the body selected from the group consisting of finger, thumb, toe, wrist, elbow, knee, ankle, foot, hand, palm and face.

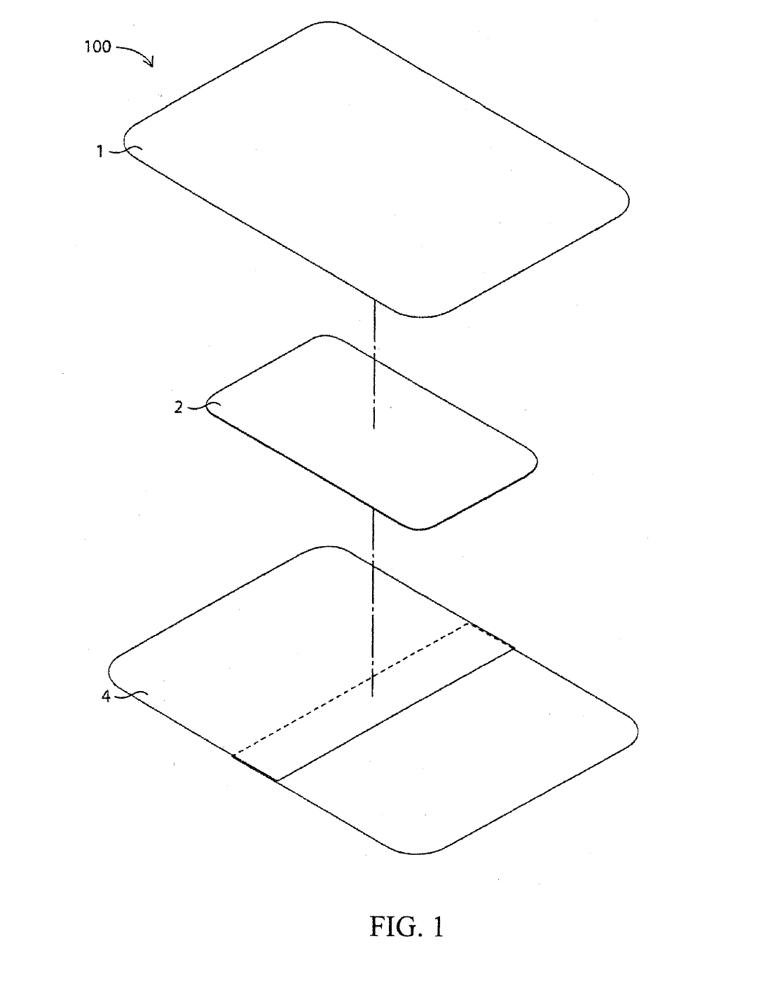

FIG. 1 depicts an expanded view of the components of one embodiment of the inventive bandage 100. Top adhesive layer 1 has is substantially rectangular with rounded corners. Top adhesive layer 1 has adhesive material disposed on the bottom surface and a top surface that is adhesive-free. The top surface of top adhesive layer 1 may include text and graphics printed on the surface. Underneath the top adhesive layer 1 is the infused hydrogel layer 2. Infused hydrogel layer 2 is sized to be smaller than top adhesive layer 1 so that top adhesive layer 1 completely covers infused hydrogel layer 2. Bandage-backing layer 4 is disposed across the entire bottom surface of the inventive bandage 100 and is sized to be slightly larger than, and substantially the same shape as, top adhesive layer 1. Backing layer 4 comprises two partially overlapping sheets—the two sheets are sized and oriented to ensure complete coverage of the inventive bandage 100 whose largest surface is top layer 1.

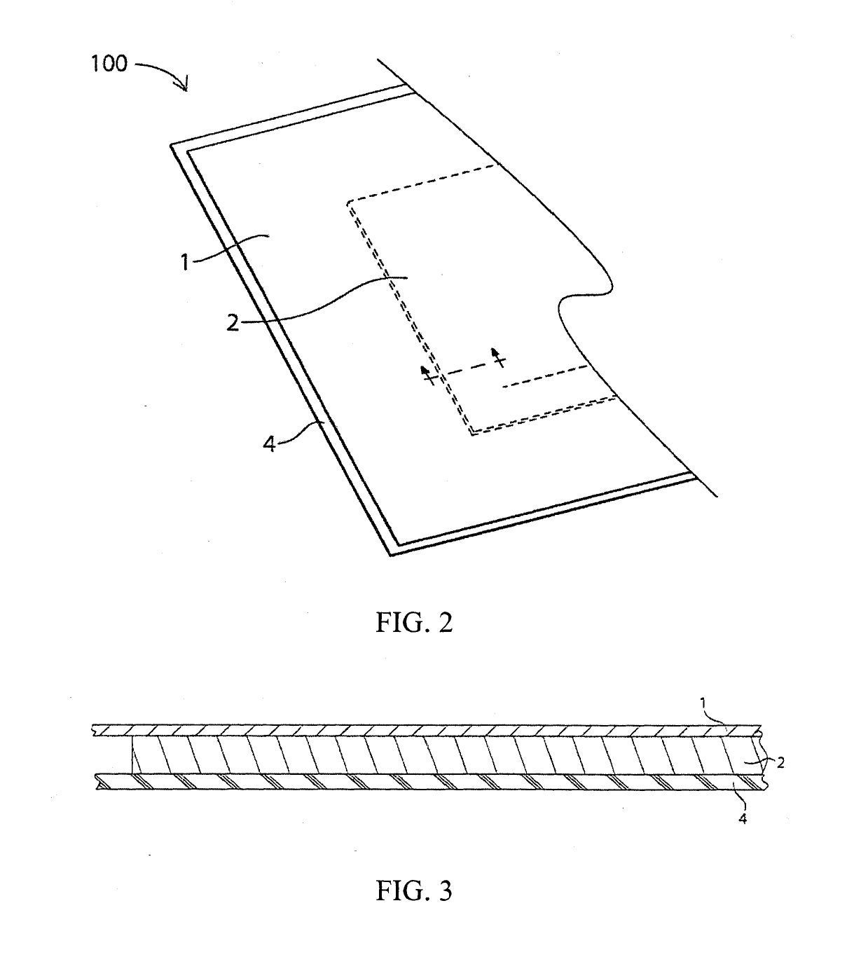

FIG. 2 depicts a schematic of one embodiment of the inventive bandage 100 showing the relative positions of top adhesive layer 1 and infused hydrogel layer 2, along with backing layer 4. In the inventive bandage 100, the adhesive surface of top adhesive layer 1 is coupled to the top side of infused hydrogel layer 2, and the inventive bandage 100 further includes a removable backing layer 4 coupled to the bottom surface of the bandage.

FIG. 3 depicts a cross-sectional view of the inventive bandage 100 of FIG. 2. As shown in FIG. 3, the entire top side of infused hydrogel layer 2 is in contact with the bottom side of adhesive layer 1. Backing layer 4 is depicted as contacting the bottom side of infused hydrogel layer 2 as well as a portion of the bottom side of adhesive layer 1.

### Further Embodiments

The inventive bandages optionally contain a thin substrate of a thermally conductive metal. Various metals or alloys may be used in the inventive bandages and preferred metals or alloys are those with efficient heat-transfer qualities. Metals or metal alloys may also be chosen based on additional qualities such as biocompatibility, chemical reactivity, or machinability. A particularly preferred metal aluminum because of its thermal conductivity.

The conductive metal layer is preferably coupled to the hydrogel so that the hydrogel is positioned in between the metal layer (bottom) and the adhesive layer (top). The conductive metal layer and infused hydrogel layer may be bonded together by the adhesive properties of the hydrogel and may also be bonded together by the addition of an adhesive.

Preferred thermally conductive metals include aluminum, silver, gold, copper, zinc, magnesium, tungsten, titanium, and platinum. Other preferred metals include iron, nickel, zinc, tin, and palladium. In one preferred embodiment the metal is aluminum. Preferably the metal contains 98.00% minimum aluminum. In one embodiment aluminum ASTM B479 1145 is used due to its ease of procurement in sizeable manufacturing quantity.

Alloys substantially based on these metals and other biocompatible metal alloys may also be used. Such alloys include aluminum alloys, chromium/molybdenum/iron alloys, or aluminum/magnesium alloys. One preferred aluminum alloy contains at least about 90% aluminum. One preferred aluminum alloy contains at least 92% aluminum and about 5% magnesium. Other metals can be used in specific quantities to fulfill a specific requirement of wound care.

One layer of metal or more than one layer of metal suitably bonded may be used in the metal substrate. In one embodiment a layer of aluminum and a layer of copper are bonded to form the thermally conductive layer. In one embodiment a layer of aluminum-clad copper is used.

The metal or metal alloy in the invention is preferably sized as a thin sheet or foil. As the metal thickness is increased, conductive performance is reduced. Additionally, as the metal thickness is increased, the bandage will increase in rigidity due to the increased force required for deformation. However, as the metal thickness is reduced, machinability and foil integrity may be reduced. The metal or metal alloy in the inventive bandage may be annealed to enhance the ductility and flexibility of the metal layer.

The metal or metal alloy preferably has a thickness in the range from about 0.00025 inches to about 0.006 inches. In one embodiment the metal or metal alloy layer is about 0.0005 inches to about 0.005 inches thick. The metal may be about 0.0005 inches, about 0.0010 inches, about 0.0015 inches, about 0.0020 inches, about 0.0025 inches, about 0.0030 inches, about 0.0035 inches, about 0.0040 inches, about 0.0045 inches or about 0.0050 inches thick. In one embodiment, the metal is about 0.0005 inches thick. In one embodiment, the metal is about 0.0020 inches thick. In a preferred embodiment, the metal is about 0.0010 inches thick. In one embodiment, the metal substrate layer is about 0.0010 inches thick.

In one embodiment the metal or metal alloy layer is substantially flat. In another embodiment the metal or metal alloy layer is textured to increase the surface area of metal in contact with the heat-sink and thus increase the efficiency of heat transfer. In one embodiment the metal layer is an aluminum sheet or foil. In one embodiment the metal layer is a sheet that has on one side a substantially smooth surface; in one embodiment the metal layer is a sheet that has on one side a dull, matte or brushed surface. In one embodiment the metal layer is an aluminum sheet that has on one side a textured surface having a plurality of discrete protrusions as depicted in FIGS. 9A-9B, 10A-10I, 11B, 12A-12B of U.S. Pat. No. 8,530,720 to Freer, et al.

In an embodiment where the metal layer is a substantially smooth sheet or foil, the metal substrate has a thickness in the range from about 0.00025 inches to about 0.006 inches. In an embodiment where the metal layer has a plurality of discrete protrusions, the metal substrate has a thickness of about 0.00025 inches to about 0.040 inches as measured from the bottom side of the metal substrate to the average peak height of the plurality of protrusions on the top side of the metal substrate.

In a preferred embodiment the infused hydrogel substrate is sized larger than the metal substrate; in a preferred embodiment the perimeter of the infused hydrogel layer completely surrounds the perimeter of the metal layer. Ideally, the metal heat spreader is designed to transfer heat from a burn wound that has considerably smaller surface area when compared to that of the bandage. The metal layer spreads the elevated burn's added heat across the entire surface of the infused hydro gel layer providing greater surface area for conduction contact and, in turn, reduced time until thermal equilibrium is reached between the burn and the hydrogel. This benefit reduces the burn temperature swiftly without significantly affecting the equilibrium temperature. Further, when a bandage that is sized larger than the size of a burn wound is applied to the burn, the time required to reach thermal equilibrium is reduced as a result of lateral heat propagation.

In a preferred embodiment, the metal layer is sized to completely cover the burn to avoid direct contact of the hydrogel to the burn area. Such a bandage would eliminate any negative adhesive properties of applying an infused hydrogel directly to a burn. Such a bandage would further benefit from the thermal conduction aspects of aluminum for heat-spreading purposes. Such a bandage would effectively cool a subject's burn and further alleviate pain associated with subject's burn.

The infused hydrogel substrate may be about 1.1 times to about 3.0 times the size of the metal layer substrate. The infused hydrogel substrate may be about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0 times the size of the metal layer substrate.

In one embodiment, the ratio of the area of the infused hydrogel substrate to the area of the metal substrate is about 3.36:2.00—where the infused hydrogel substrate is about 1.68 times larger than the metal substrate. In one embodiment, the ratio of the area of the infused hydrogel substrate to the area of the metal substrate is about 8.16:6.00—where the infused hydrogel substrate is about 1.36 times larger than the metal substrate. In one embodiment, the ratio of the area of the infused hydrogel substrate to the area of the metal substrate is about 12.76:10.00—where the infused hydrogel substrate is about 1.28 times larger than the metal substrate. In one embodiment, the ratio of the area of the infused hydrogel substrate to the area of the metal substrate is about 1.11:0.45—where the infused hydrogel substrate is about 2.46 times larger than the metal substrate. In one embodiment, the ratio of the area of the infused hydrogel substrate to the area of the metal substrate is about 1.28:0.56—where the infused hydrogel substrate is about 2.27 times larger than the metal substrate. In one example the inventive bandage includes a substantially rectangular metal layer having dimensions of about 2.00 inches by about 1.00 inches, and substantially rectangular infused hydrogel layer having dimensions of about 2.40 inches by about 1.4 inches.

In one embodiment, the top adhesive layer, infused hydrogel substrate and metal substrate of the inventive bandage are concentric to one another. In another embodiment, the infused hydrogel substrate and metal substrate are concentric to each other and are positioned so as to be off-center from the top adhesive layer within the inventive bandage. In one embodiment the entire top surface of the infused hydrogel substrate is in contact with the bottom surface of the top adhesive layer; in one embodiment the entire top surface of the metal substrate is in contact with the bottom surface of the infused hydrogel substrate. In one embodiment the backing layer is in contact with the bottom surface of the inventive bandage such that the backing layer contacts a portion of the bottom surface of the top adhesive layer, a portion of the bottom surface of the infused hydrogel substrate, and the entire bottom surface of the metal substrate.

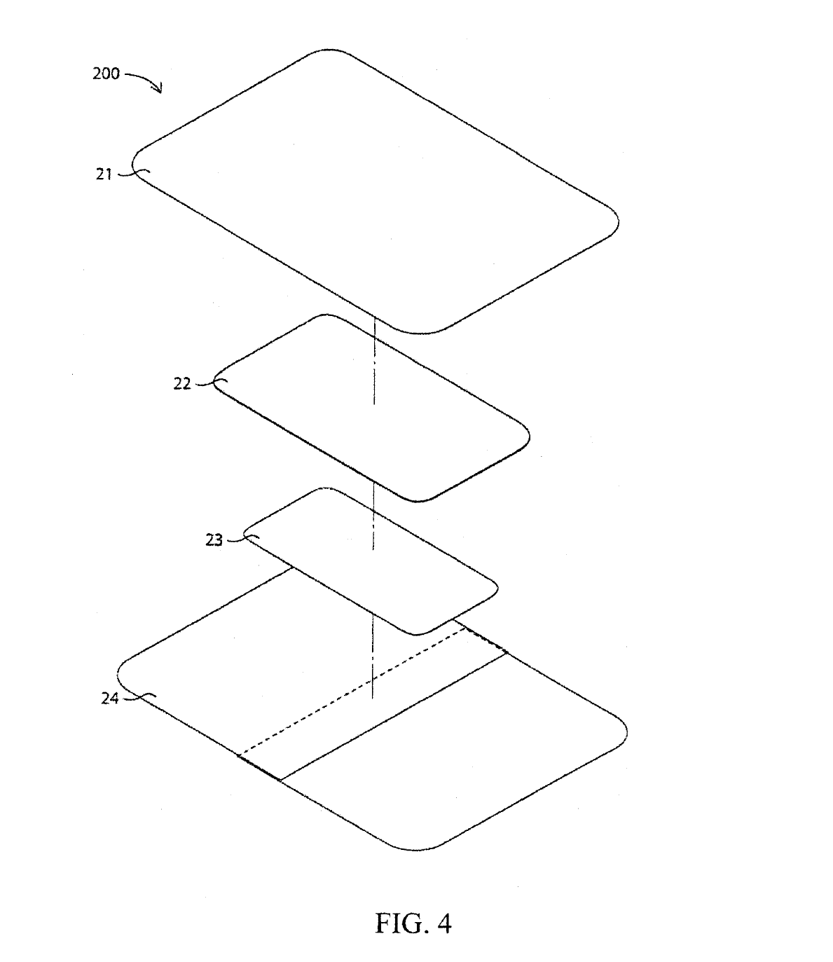

FIG. 4 depicts an expanded view of the components of one embodiment of the inventive bandage 200. Top adhesive layer 21 has is substantially rectangular with rounded corners. Top adhesive layer 21 has adhesive material disposed on the bottom surface and a top surface that is adhesive-free. The top surface of top adhesive layer 21 may include text and graphics printed on the surface. Underneath the top adhesive layer 21 is the infused hydrogel layer 22. Infused hydrogel layer 22 is sized to be smaller than top adhesive layer 21 so that top adhesive layer 21 completely covers infused hydrogel layer 22. Underneath infused hydrogel layer 22 is the thermally conductive metal layer 23. Metal layer 23 is sized to be smaller than infused hydrogel layer 22 so that infused hydrogel layer 22 completely covers metal layer 23. Bandage-backing layer 24 is disposed across the entire bottom surface of the inventive bandage 200 and is sized to be slightly larger than, and substantially the same shape as, top adhesive layer 21. Backing layer 24 comprises two partially overlapping sheets—the two sheets are sized and oriented to ensure complete coverage of the inventive bandage 200 whose largest surface is top layer 21.

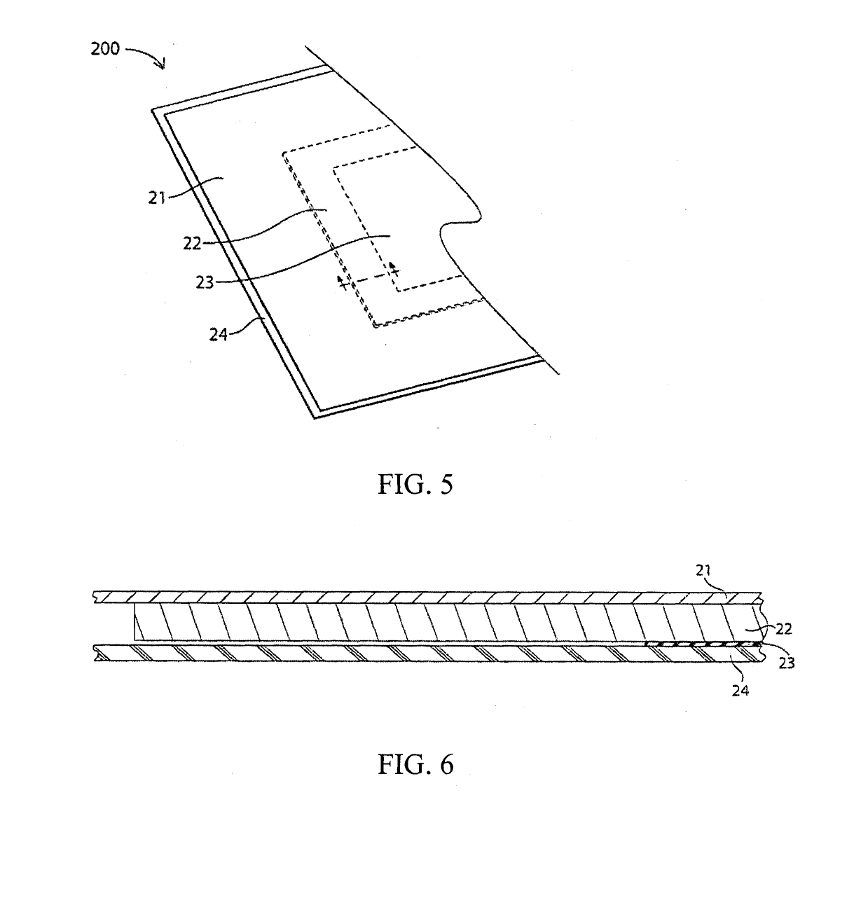

FIG. 5 depicts a schematic of one embodiment of the inventive bandage 200 showing the relative positions of top adhesive layer 21, infused hydrogel layer 22, and metal layer 23, along with backing layer 24. In the inventive bandage 200, the adhesive surface of top adhesive layer 21 is coupled to the top side of infused hydrogel layer 22; the bottom side of infused hydrogel layer 22 is coupled to the top side of the metal layer 23; and the inventive bandage 200 further includes a removable backing layer 24 coupled to the bottom surface of the bandage.

FIG. 6 depicts a cross-sectional view of the inventive bandage 200 of FIG. 5. As shown in FIG. 6, the entire top side of infused hydrogel layer 22 is in contact with the bottom side of adhesive layer 21. Further, the entire top side of metal layer 23 is in contact with the bottom side of infused hydrogel layer 22. Backing layer 24 is depicted as contacting the bottom side of metal layer 23, but backing layer 24 will also contact a portion of the bottom side of infused hydrogel layer 22 as well as a portion of the bottom side of adhesive layer 21.

The inventive bandage can be further enhanced by the inclusion of a thermochromic indicator member, wherein the thermochromic indicator member is in thermal communication with a burn wound via the top adhesive layer. A thermochromic compound—similar to what is typically found in mood rings—provides visual feedback regarding the heat removed from the subject's burn. The thermochromic indicator member is comprised of material calibrated to indicate when a burn on which said bandage is applied is still too warm for safe removal of said bandage, based on a predetermined threshold, and indicate when a burn has cooled to at least a predetermined threshold such that said bandage can be safely removed and/or changed-out for a new medical dressing.

In one embodiment the thermochromic indicator member provides color-based indications as to the thermal status of the burn to which said bandage is applied. In another embodiment the thermochromic indicator member provides icon-based indications as to the thermal status of the burn to which the bandage is applied. In some applications, the thermochromic indicator member is comprised of material selected from the group consisting of thermochromic liquid crystals, leuco dyes, and thermochromic inks.

In one embodiment a metal substrate has an extended member that extends beyond the border of the coupled infused hydrogel layer to be under, and in direct contact with the thermochromic compound present in the top adhesive layer such that the metal extension provides thermal communication between a burn and the thermochromic compound. In one embodiment the thermochromic indicators have compounds calibrated to indicate when a burn is sufficiently cooled (for example by providing a color indicator such as green and/or an icon indicator such as a happy face) or still too warm (for example by providing a color indicator such as red and/or an icon indicator such as sad face). In one embodiment the inventive bandage has a thermochromic compound that does not present a visible color at room temperature; upon application of the bandage to a burn the thermochromic compound turns red (indicating the subject should keep the bandage in place); after time passes and the burned tissue cools the thermochromic compound turns green (indicating the subject may remove the bandage).

In one embodiment the thermochromic indicator changes color on the end closest to the metal substrate more quickly than the end farthest from the metal substrate due to a temperature gradient across the indicator. Stratification of the color change of the thermochromic indicator provides indication regarding the rate and amount of cooling.

Additional components may also be included with the bandage such as antibacterial agents to suppress bacterial growth, biomoloecules such as growth factors and protease inhibitors assisting with wound healing or anesthetics and analgesics to reduce pain. Antibacterial agents may include metal ions (such as silver ions) or metal salts (such as silver nitrate, lactate or citrate, or aluminum diacetate), metal nanoparticles (such as silver nanoparticles), sulfates and silvers, antibacterial peptides, quaternary ammonium compounds, triclosan, iodine, PVP-iodine, phenol compounds, chlorhexidine gluconate, polyhexamide, silver sulfadiazine, octenidine, as well as antibiotics such as sulfate, beta-lactams, fluoroquinolones, aminoglycosides, glycopeptides, oxazolidinones, bacteriocin, or tetracycline. Growth factors may include platelet derived growth factor (PDGF), fibroblast growth factor (FGF), epidermal growth factor (EGF), and vascular endothelial growth factor (VEGF). Anesthetics and analgesics may include lidocaine, benzocaine, procaine, aloe, menthol, paracetamol, non-steroidal anti-inflammatory drugs and opioid drugs. In one embodiment heparan sulfate is included in the bandage as a promoter of wound healing. In one embodiment heparan derived glycosaminoglycans including dermatan sulfate, keratan sulfate, chondroitin-4 and chondroitin-6-sulfate, and hyaluronic acid may be added to accelerate wound healing.

Depending on the type and severity of burn, in addition to the inventive bandage a thermally conductive adhesive, paste, gel, or grease may be applied to the area of a subject's skin to enhance the heat transfer from a burn wound to the thermally conductive metal layer. In some of these variations, the thermally conductive compound is derived from metal or silicone (usually with a zinc-oxide or aluminum-oxide inclusion to improve conductivity), and may fill gaps where air would normally be present. The thermally conductive compound provides a superior conductor (as compared to air) almost equal to that of the conductor itself. The performance of thermally conductive compound is measured in W/m-K. Standard silicon/zinc-oxide thermal compound has thermal conductivities in the range of 0.7-0.9 W/m-K. In such variations, the thermally conductive medium used can also be an aluminum-infused medicinal/therapeutic cream, ointment, or other compound.

While the present inventions have been illustrated and described in many embodiments of varying scope, it will at once be apparent to those skilled in the art that variations may be made within the spirit and scope of the inventions. Accordingly, it is intended that the scope of the inventions set forth in the appended claims not be limited by any specific wording in the foregoing description, except as expressly provided.

### Examples

### Example 1

The following example is meant to be illustrative and prophetic only. In this example, an inventive bandage is comprised of a top adhesive layer, a middle hydrogel layer infused with aluminum oxide particles having a mean size of approximately 10 micrometers, and a bottom backing layer. The top adhesive layer is substantially rectangular and has dimensions of about 3.4 inches by about 2.4 inches with a thickness of about 0.0044 inches. The top adhesive layer is made of commercially available medical tape.

Coupled to the top adhesive layer is a middle hydrogel layer infused with aluminum oxide particles that is substantially rectangular having dimensions of about 2.3 inches by about 1.3 inches with a thickness of about 0.015 inches.

Finally a bottom backing layer is coupled to the bandage. The backing layer is substantially rectangular having dimensions of about 3.4 inches by about 2.4 inches with a thickness of about 0.0061 inches. The backing layer is comprised of two equally sized sheets each about 1.9 inches by about 2.4 inches—the sheets overlap each other by about 0.5 inches to facilitate removal from the bandage.

### Example 2

The following example is meant to be illustrative and prophetic only. In this example, an inventive bandage of Example 1 is applied to a burn. The aluminum oxide infused hydrogel layer draws heat away from the burn via conduction. Within about 15 to about 120 seconds, the burn is cooled by the infused hydrogel substrate, and the discomfort and pain caused by the burn are reduced.

### Example 3

The following example is meant to be illustrative and prophetic only. In this example, an inventive bandage is comprised of a top adhesive layer, a hydrogel layer infused with aluminum oxide particles having a mean size of approximately 10 micrometers, an aluminum layer, and a backing layer. The top adhesive layer is substantially rectangular and has dimensions of about 3.4 inches by about 2.4 inches with a thickness of about 0.0044 inches. The top adhesive layer is made of commercially available medical tape.

Coupled to the top adhesive layer is a hydrogel layer infused with aluminum oxide particles that is substantially rectangular having dimensions of about 2.3 inches by about 1.3 inches with a thickness of about 0.015 inches.

Coupled to the infused hydrogel layer is an aluminum layer. The aluminum layer is substantially rectangular and has dimensions of about 2.0 inches by about 1.0 inches with a thickness of about 0:001 inches. The aluminum is a sheet conforming to ASTM B479 1145.

A backing layer is coupled to the bandage. The backing layer is substantially rectangular having dimensions of about 3.4 inches by about 2.4 inches with a thickness of about 0.0061 inches. The backing layer is comprised of two equally sized sheets each about 1.9 inches by about 2.4 inches—the sheets overlap each other by about 0.5 inches to facilitate removal from the bandage.

### Example 4

The following example is meant to be illustrative and prophetic only. In this example, an inventive bandage of Example 3 is applied to a burn. The aluminum layer draws heat away from the burn via conduction and transfers the thermal energy via conduction to the hydrogel layer. Within about 15 to about 120 seconds, thermal equilibrium is reached between the burn and the infused hydrogel substrate, and the discomfort and pain caused by the burn are reduced.

The following example is meant to be illustrative and prophetic only. In this example, an inventive bandage is comprised of a top adhesive layer, a middle hydrogel layer infused with aluminum oxide particles having a mean size of approximately 10 micrometers and menthol having pain relief ability, and a bottom backing layer. The top adhesive layer is substantially rectangular and has dimensions of about 3.4 inches by about 2.4 inches with a thickness of about 0.0044 inches. The top adhesive layer is made of commercially available medical tape.

What is claimed is:

1. A bandage comprising:
(a) a top layer of polymeric material, the top layer having a first surface and a second surface, and where the second surface of the top layer has adhesive disposed thereon; and
(b) a bottom layer of hydrogel substrate, the hydrogel substrate having a first surface and a second surface, where the first surface of the hydrogel substrate is coupled to the second surface of the top layer, and where the hydrogel substrate comprises a thermally conductive material.

2. A bandage of claim 1 wherein said thermally conductive material comprises aluminum.

3. A bandage of claim 1 wherein said thermally conductive material comprises aluminum oxide particles.

4. A bandage of claim 3 wherein said aluminum oxide particles are between about 1 nanometer to about 500 micrometers in size.

5. A bandage of claim 3 wherein said aluminum oxide particles have a mean size of about 3 to about 20 micrometers.

6. A bandage of claim 3 wherein said aluminum oxide particles have a mean size of approximately 10 micrometers.

7. A bandage of claim 3 wherein said aluminum oxide particles have a mean size smaller than about 100 nanometers.

8. A bandage of claim 3 wherein said aluminum oxide particles have a mean size of approximately 10 micrometers.

9. A bandage of claim 8 wherein said aluminum oxide particles are approximately 10 micrometers.

10. A bandage of claim 3 further comprising a backing layer removably coupled to the bottom surface of the bandage.

11. A bandage of claim 1 further comprising a thermochromic indicator member disposed within the top layer.

12. A bandage of claim 3 further comprising (c) a conductive metal layer coupled to the second surface of the hydrogel substrate.

13. A bandage of claim 3 wherein the hydrogel substrate comprises polyvinylpyrrolidone.

14. A dual-layered bandage having a top surface and a bottom surface, said bandage consisting of: a top layer of a polymeric material with a top surface and a bottom surface, wherein said bottom surface has adhesive disposed thereon; and a bottom layer of a hydrogel comprising a thermally conductive material.

15. A dual-layered bandage of claim 13 wherein said thermally conductive material comprises aluminum or aluminum oxide particles.

16. A bandage of claim 13 wherein said thermally conductive material comprises aluminum oxide particles between about 1 nanometer to about 500 micrometers in size.

17. A bandage of claim 13 wherein said thermally conductive material comprises aluminum oxide particles having a mean size of approximately 10 micrometers.

18. A method of treating a burn in a subject comprising: applying a bandage according to any of claims 1-17 to a subject's burn wherein heat is dissipated from the burn to the hydrogel substrate.

19. A method of treating a burn in a subject according to claim 18 wherein the symptoms of burn are alleviated, reduced, or eliminated.

20. A method of treating a burn in a subject according to claim 19 wherein the symptoms of burn are alleviated, reduced, or eliminated within about 15 to about 300 seconds of bandage application.

21. A bandage of claim 1 wherein said thermally conductive material comprises at least one of aluminum oxide particles, titanium dioxide particle, and zinc oxide particles.

22. A bandage of claim 1 wherein said hydrogel substrate comprises about 10% thermally conductive material.

23. A bandage of claim 1 wherein said hydrogel substrate is comprised of (a) about 1% to about 70% monomer; (b) about 0.01% to about 20% cross-linker; (c) about 0.001% to about 1% photoinitiator; (d) about 1% to about 40% thermally conductive material; and (e) about 1% to about 30% humectant.

24. A bandage of claim 23 comprising about 30% acrylamide.

25. A bandage of claim 23 comprising about 0.1% methylene-bisacrylamide.

26. A bandage of claim 23 where said photoinitiator comprises about 0.08% 1-[4-(2-Hydroxyethoxy)-phenyl]-2-hydroxy-2-methyl-1-propane-1-one.

27. A bandage of claim 23 comprising about 10% aluminum oxide.

28. A bandage of claim 23 comprising about 10% glycerol.

29. A bandage of claim 23 comprising (a) about 30% acrylamide; (b) about 0.1% methylene-bisacrylamide; (c) about 0.08% 1-[4-(2-Hydroxyethoxy)-phenyl]-2-hydroxy-2-methyl-1-propane-1-one; (d) about 10% aluminum oxide; and (e) about 10% glycerol.

30. A bandage of claim 1 wherein said hydrogel substrate is comprised of (a) about 1% to about 30% polymer; (b) about 0.01% to about 20% cross-linker; (c) about 0.001% to about 0.2% photoinitiator; (d) about 1% to about 40% thermally conductive material; and (e) about 1% to about 30% humectant.

31. A bandage of claim 30 comprising about 10% polyvinylpyrrolidone.

32. A bandage of claim 30 where said cross-linker comprises about 2% polyethylene glycol diacrylate.

33. A bandage of claim 30 where said photoinitiator comprises about 0.1% 1-[4-(2-Hydroxyethoxy)-phenyl]-2-hydroxy-2-methyl-1-propane-1-one.

34. A bandage of claim 30 comprising about 10% aluminum oxide.

35. A bandage of claim 30 comprising about 10% glycerol.

36. A bandage of claim 30 comprising (a) about 10% polyvinylpyrrolidone; (b) about 2% polyethylene glycol diacrylate; (c) about 0.1% 1-[4-(2-Hydroxyethoxy)-phenyl]-2-hydroxy-2-methyl-1-propane-1-one; (d) about 10% aluminum oxide; and (e) about 10% glycerol.

37. A bandage of claim 23 comprising (a) about 40% 2-acrylamido-2-methylpropane sulfonic acid; (b) about 0.2% polyethylene glycol diacrylate; (c) about 0.05% 1-[4-(2-Hydroxyethoxy)-phenyl]-2-hydroxy-2-methyl-1-propane-1-one; (d) about 10% aluminum oxide; and (e) about 10% glycerol.

38. A bandage of any of claim 3-10, 12-13, 15-17, 21, 27, 29, 34 or 37 wherein said aluminum oxide is comprised of a mixture of particles of different sizes.
